(19) United States
(12) Patent Application Publication
(10) Pub. No.: US 2026/0011039 A1
(43) Pub. Date: Jan. 8, 2026

(54) GENERATION OF IMAGE SETS FOR COGNITIVE ASSESSMENT

(71) Applicant: Cumulus Neuroscience Ltd., Belfast (IE)

(72) Inventors: George Stothart, Frome (GB); Brian Edmond Murphy, Kilcoole (IE)

(21) Appl. No.: 18/764,056

(22) Filed: Jul. 3, 2024

(51) Int. Cl. G06T 11/00; A61B 5/378; G06T 7/00

(52) U.S. Cl. CPC G06T 11/00; G06T 7/0002; A61B 5/378

(57) ABSTRACT

Techniques for generating image sets for cognitive assessment are provided. A first machine learning (ML) model may generate a plurality of candidate images that are each expected to meet a set of visual and semantic criteria. Each of the plurality of candidate images that meets the set of visual and semantic criteria may be identified as a test image. A second ML model may be used to generate a set of test images using the plurality of test images by adding to the set of test images, test images whose visual and semantic properties are sufficiently different from the visual and semantic properties of each other test image currently in the set of test images.

### TECHNICAL FIELD

The present disclosure relates generally to image generation for cognitive assessment, and more particularly, to use of machine learning and other algorithms to generate image sets that are suitable for cognitive assessments such as those that used electroencephalography (EEG) as a measurement technique.

### BACKGROUND

A patient's memory and cognitive performance can be assessed by presenting a set of standardized images to the patient and recording electrical signals corresponding the patient's brain activity while the set of images is being presented. The assessment relies on the patient seeing the set of images for the very first time, encoding those images in memory, and then passively recognizing that some of those images (often referred to as “key” images) are presented repeatedly. The images used for such assessments should have certain characteristics such as a white background, a clear unitary object of interest, easily differentiable, and easy to parse visually, among others.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a block diagram depicting an example environment in which techniques for image set generation for cognitive assessment can be performed, according to some embodiments of the present disclosure.

FIG. 2 is a detailed block diagram of the different components of an image set generation module, according to some embodiments of the present disclosure.

FIG. 3 is a block diagram illustrating the functionality of an image generation component of the image set generation module of FIG. 2, according to some embodiments of the present disclosure.

FIG. 4 is a block diagram illustrating the functionality of a filtering component of the image set generation module of FIG. 2, according to some embodiments of the present disclosure.

FIGS. 5A-5C are block diagrams illustrating the functionality of an image set making component of the image set generation module of FIG. 2, according to some embodiments of the present disclosure.

FIG. 5D is a detailed block diagram of the different components of an image set generation module, according to some embodiments of the present disclosure.

FIG. 6 is a flow diagram depicting a method of generating image sets for cognitive assessment, according to some embodiments of the present disclosure.

FIG. 7 is a block diagram of an example computing device that may perform one or more of the operations described herein, in accordance with some embodiments.

### DETAILED DESCRIPTION

Currently, image sets for cognitive and memory assessment are prepared and curated by hand. Proper assessment of cognitive and memory functions relies upon participants being presented with “fresh” images that they have never seen before the assessment. If images are re-used (e.g., from previous assessments), this will compromise the accuracy of the results. In practice, this makes repeated assessment at periodic visits difficult. It can be difficult to track improvement or decline over a series of assessments, and in cases where a first attempted assessment needed to be aborted, obtaining accurate results using the same images during a subsequent assessment will compromise the assessment. There is also a risk that if the set of images used for an assessment is widely used, some of the images could “leak” into the public domain resulting a patient knowing in advance which images are the key images, and compromising the accuracy of the results.

Aspects of the present disclosure address the above-noted and other deficiencies by providing techniques for generation of image sets for cognitive assessment. A first machine learning (ML) model may generate a plurality of candidate images that are each expected to meet a set of visual and semantic criteria. Each of the plurality of candidate images that meets the set of visual and semantic criteria may be identified as a test image. A second ML model may be used to generate a set of test images using the plurality of test images by adding a first test image from the plurality of test images to the set of test images. For each subsequent test image of the plurality of test images, that second ML model may add the subsequent test image to one or more sets of test images if it is determined that a difference between visual and semantic properties of the subsequent test image and visual and semantic properties of each other test image currently in the set of test images is sufficiently large (i.e., meets a threshold) based on a set of distance metrics.

The second ML model may generate a number of test image sets using the plurality of test images. Once the test image sets are generated, they may be stored and used to assess a patient. For example, a first test image set may be used during a first assessment, and a new test image set may be used for each subsequent assessment, allowing for longitudinal tracking of progression, such as response to a therapy or treatment. Having a number of test image sets available also increases the precision of detection/diagnosis since there is more statistical power behind such detection/diagnosis due to both the raw increase in evidence and the ability to repeat assessments over different time periods (either aggregating multiple samples, each with random noise (e.g. “good” days, and “bad” days), in the same time period; or taking periodic samples to track change over time).

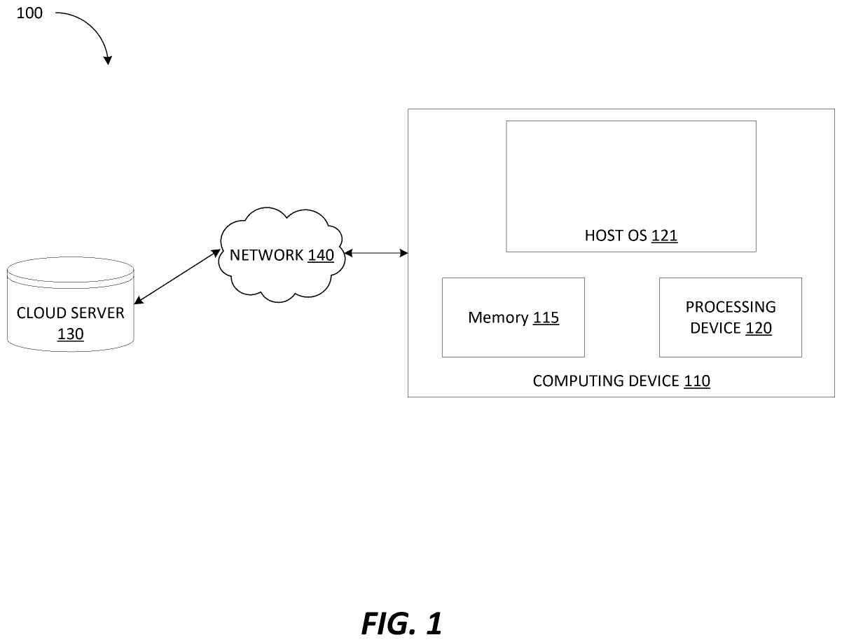

FIG. 1 is a block diagram depicting an example environment 100 in which some embodiments of the present disclosure may be implemented. Environment 100 includes a computing device 110, a cloud service 130, and a network 140. The computing device 110 and the cloud service 130 may be coupled to each other (e.g., may be operatively coupled, communicatively coupled, may communicate data/messages with each other) via network 140. Network 140 may be a public network (e.g., the internet), a private network (e.g., a local area network (LAN) or wide area network (WAN)), or a combination thereof. In one embodiment, network 140 may include a wired or a wireless infrastructure, which may be provided by one or more wireless communications systems, such as a WiFi™ hotspot connected with the network 140 and/or a wireless carrier system that can be implemented using various data processing equipment, communication towers (e.g., cell towers), etc. The network 140 may carry communications (e.g., data, message, packets, frames, etc.) between computing device 110 and cloud service 130. The computing device 110 and cloud service 130 may include hardware such as processing device 120 (e.g., processors, central processing units (CPUs), memory 115 (e.g., random access memory (RAM), storage devices (e.g., hard-disk drive (HDD), solid-state drive (SSD), etc.), and other hardware devices (e.g., sound card, video card, etc.). A storage device may comprise a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices.

FIG. 1 and the other figures may use like reference numerals to identify like elements. A letter after a reference numeral, such as “110A,” indicates that the text refers specifically to the element having that particular reference numeral. A reference numeral in the text without a following letter, such as “110,” refers to any or all of the elements in the figures bearing that reference numeral.

The computing device 110 and cloud service 130 may each comprise any suitable type of computing device or machine that has a programmable processor including, for example, server computers, desktop computers, laptop computers, tablet computers, smartphones, set-top boxes, etc. In some examples, the computing device 110 and cloud service 130 may each comprise a single machine or may include multiple interconnected machines (e.g., multiple servers configured in a cluster). The computing device 110 and cloud service 130 may be implemented by a common entity/organization or may be implemented by different entities/organizations. For example, computing device 110 may be operated by a first company/corporation and cloud service 130 may be operated by a second company/corporation. The computing device 110 may execute or include an operating system (OS), as discussed in more detail below. The host OS 121 of computing device 110 may manage the execution of other components (e.g., software, applications, etc.) and/or may manage access to the hardware (e.g., processors, memory, storage devices etc.) of their respective computing device.

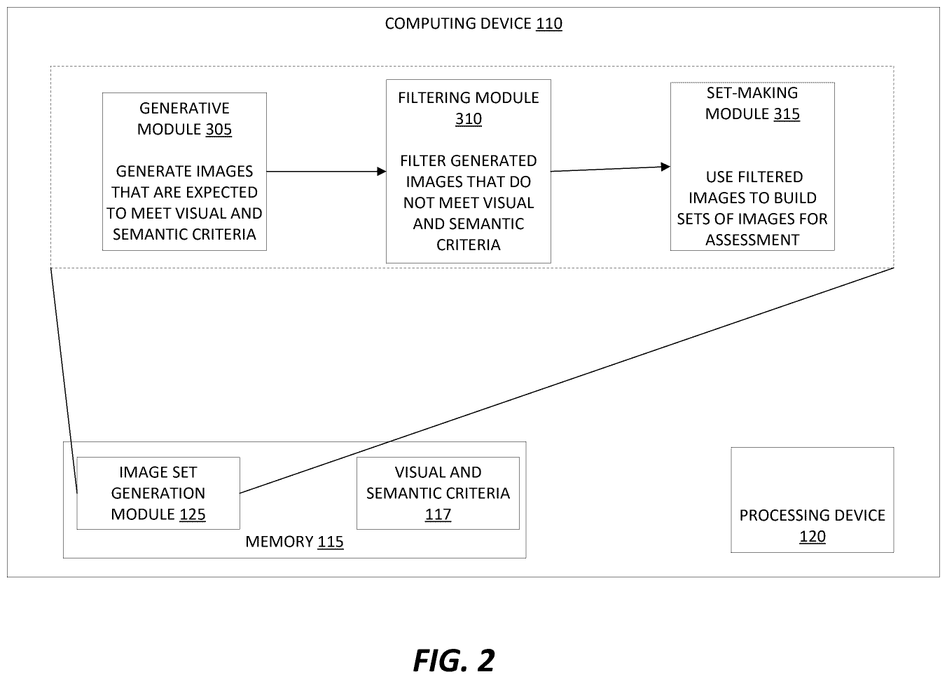

FIG. 2 illustrates an image set generation module 125, which may be stored in memory 115 and may comprise software or logic which can be executed by processing device 120 to perform the techniques described herein for image set generation. As shown in FIG. 2, the image set generation module 125 may comprise three components, a generative module 305, a filtering module 310 and a set making module 315. As discussed herein, a cognitive assessment session involves a series of standardized images being presented to a patient being assessed. As the participant sees each image, the image may be encoded in their memory and then they may passively recognize those images that are repeated (key images). It should be noted that the image sets generated using the techniques described herein may also be used for active memory assessments as well. Each image used in a cognitive assessment image set should meet a set of visual and semantic characteristics, which are stored in the memory 115 as visual and semantic criteria 117. Example characteristics included in the visual and semantic criteria 117 include:
A plain/nondescript and generic background (e.g., a white background or other background that is relatively featureless)
A clear unitary object of interest whose semantics can be easily determined (e.g., is an electronic device, or a pet, or a piece of kitchenware).
A certain position/orientation of the unitary object of interest
The unitary object of interest should be able to be named out loud by the patient
The unitary object of interest should not contain human or animal faces, text, aversive subject matter (e.g., spider or monster) or offensive subject matter (e.g., nudity)
The unitary object of interest should be easily differentiable
The unitary object of interest should be reasonably memorable
The unitary object of interest should be easy to parse visually and not require especially high visual acuity
The unitary object of interest should not be culturally specific (as this may compromise understanding by people from different national backgrounds).

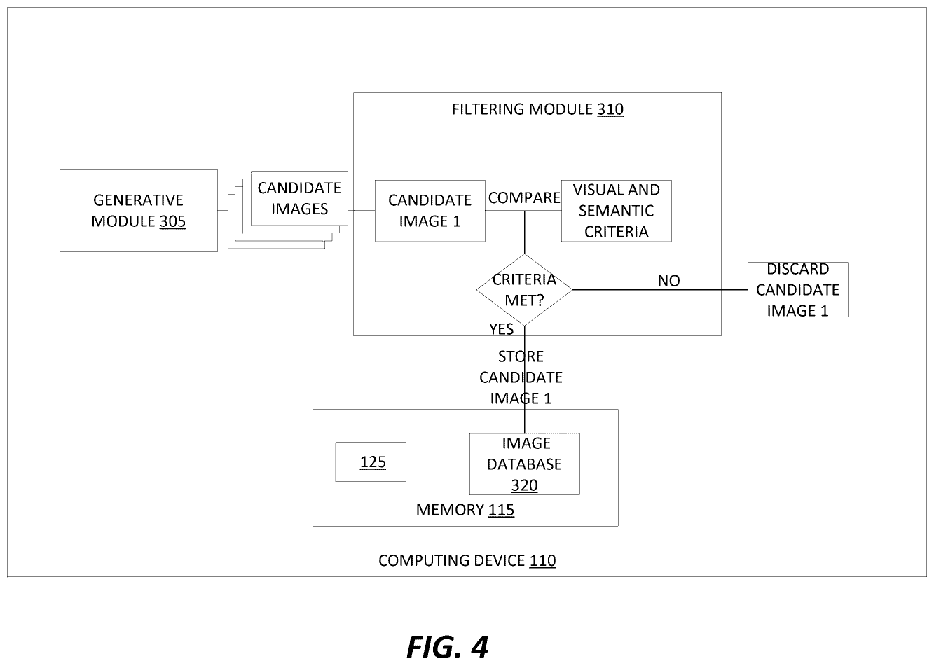

The generative module 305 may generate candidate images that are expected to meet the visual and semantic criteria 117. Each candidate image generated by the generative module 305 may be analyzed by the filtering ML module 310 to determine whether it meets the visual and semantic criteria 117. Candidate images that meet the visual and semantic criteria 117 may be stored in a database 320 (shown in FIG. 4) as test images. The set making module 315 may retrieve test images from the database 320 and use them to build test image sets.

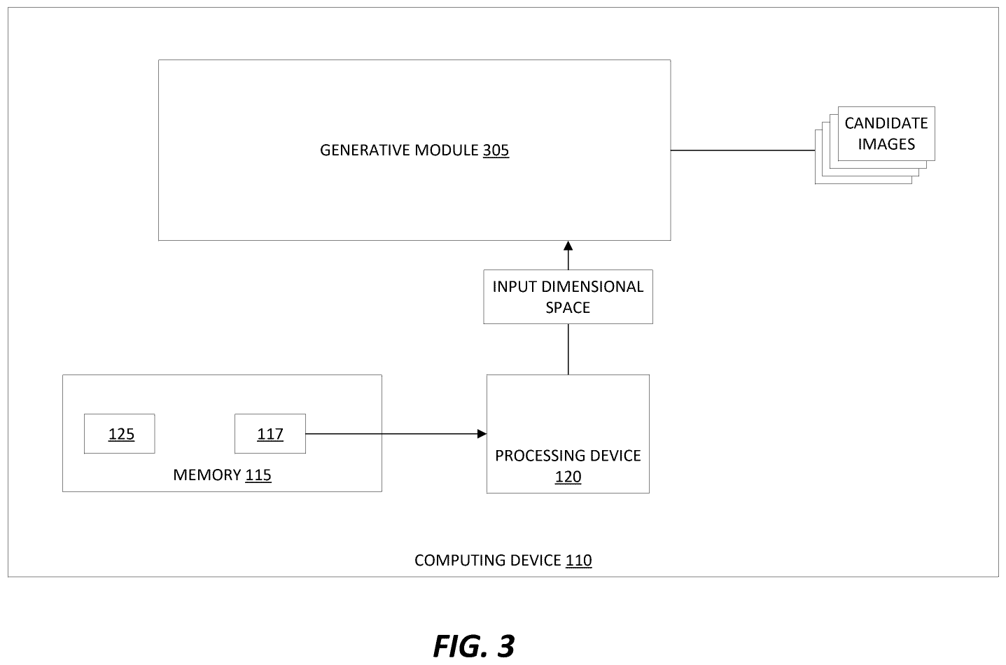

FIG. 3 illustrates the operation of the generative module 305. In some embodiments, the generative module 305 may comprise any appropriate image generation ML model that has been pretrained using a variety of image and corresponding text data to generate broad domain images based on certain input stimulus such as a set of sample images and/or text descriptions. In one example, the generative module 305 may be a variational autoencoder (VAEs), which is a probabilistic model that encodes images into a latent space, where they are represented as vectors. A decoder then reconstructs the images from the encoded vectors, enabling the model to generate new images by sampling from the latent space. In another example, the generative module 305 may be a Generative Adversarial Network (GANs), which comprises two neural networks, a generator and a discriminator, engaged in a competitive process. The generator creates synthetic images to fool the discriminator, which in turn aims to distinguish between real and fake images. This back-and-forth process between the generator and the discriminator results in the generation of highly realistic images. In some embodiments, the generative module 305 may also include natural language processing (NLP) models/utilize NLP techniques to enable the conversion of input text descriptions into corresponding visual representations.

During training, the generative module 305 may be trained to capture the semantic and visual aspects of the training images, such as the identity of the central object, and shapes, textures, colors, and styles. Any appropriate feature-based metrics, such as inception score (IS), Fréchet inception distance (FID), and perceptual path length (PPL) can be used to compare the feature distributions of the generated and training images to assess how well the generative module 305 preserves the diversity and quality of the original training domain.

The processing device 120 may generate an input dimensional space for the generative module 305 based on the visual and semantic criteria 117 and other factors. More specifically, the processing device 120 may generate an input dimensional space that specifies generation of images that meet the visual and semantic criteria 117, specifies a base level of semantic and visual difference that must exist between all images, specifies a first heightened level of semantic and visual difference that must exist between key images and distractor images, and specifies a second heightened level of semantic and visual difference that must exist between key images. In some embodiments, the processing device 120 may include as part of the input dimensional space, a list of candidate objects of interest which the generative module 305 will also use to create images. Because it is important for images used in a test image set to have a base level of visual and semantic difference from each other, the processing device 120 may specify (e.g., as text that is included within the input dimensional space) that when creating an new image using an object of interest that has been used to create one or more previous images, the generative module 305 should change certain visual properties of the object of interest (e.g., color, position, orientation) when creating the new image so that the new image differs sufficiently from the one or more previous images that include the same object of interest. The processing device 120 may also specify a (user-provided) ratio of key images to distractor images in the input dimensional space so that the generative module 305 can generate a candidate key image for every X candidate distractor images it generates based on the ratio. The ratio of key images to distractor images may correspond to the ratio of key images to distractor images required in a test image set.

As discussed herein, a test image set may include a large number of distractor images which are only presented to the patient once during assessment, as well as a relatively smaller number of key images which may be presented to the patient multiple times during the assessment for passive recognition. It is important for assessment purposes that key images have a heightened level of visual and semantic difference from distractor images that goes beyond the base level of visual and semantic difference that needs to exist between all generated images. In addition, key images must have a level of visual and semantic difference from each other that also goes beyond the base level of visual and semantic difference that needs to exist between all generated images. To do this, the processing device 120 may define e.g., as text that is included within the input dimensional space, the base level of semantic and visual difference that must exist between all images, the first heightened level of semantic and visual difference that must exist between key images and distractor images, and the second heightened level of semantic and visual difference that must exist between key images.

In some embodiments, the generative module 305 may receive further training using existing test images and corresponding text descriptions that have been e.g., hand-selected to emphasize/teach the different aspects of the visual and semantic criteria 117.

Based on the input dimensional space, the generative module 305 may begin generating candidate images (including distractor images and key images) that are expected to meet the visual and semantic criteria 117 as well as the various levels of semantic and visual difference that must exist (as discussed hereinabove). The generative module 305 may identify each candidate image it generates as a key image or a distractor image.

In some embodiments, the generative module 305 may be implemented as an algorithm that does not require ML-style training. For example, the generative module 305 may comprise an algorithm that processes an existing bank of images, or images found on the web, and removes backgrounds to see if only one object remains. Such an approach is useful because what is needed are relatively large numbers of test images, and generating all of the needed test images from scratch will result in certain generated images being unsuitable (e.g., because the image is inappropriate or somehow unrealistic). Thus, the generative module 305 may be any appropriate background removal algorithm (e.g., removebg™) that may remove background details from images and provide them to the filtering module 310 (discussed herein).

As the generative module 305 generates candidate images, each candidate image may be analyzed by the filtering module 310 to determine whether the candidate image meets the visual and semantic criteria 117. The filtering module 310 may comprise any appropriate anomaly detection ML model/algorithm, and may be trained using an existing collection of training images that meet the semantic and visual criteria 117 to recognize what images that meet the semantic and visual criteria 117 should look like. For example, the filtering module 310 may encode (compress) each training image into a lower-dimensional representation, where the encoded representations capture the key features of images that meet the semantic and visual criteria 117. The filtering module 310 can then be used to reconstruct the original training image from the encoded representation. When a candidate image that does not meet the semantic and visual criteria 117 is presented, the filtering module 310 will not be able to reconstruct it properly because it has not seen that kind of image before. The reconstruction error will be higher for a candidate image that does not meet the semantic and visual criteria 117, which can be used to identify it as an outlier. In some embodiments, the collection of training images may comprise images generated by the generative module 305 which have labelled as meeting the semantic and visual criteria 117.

Although described as above, this is for example purposes only and the filtering module 310 may be implemented in any appropriate way. For example, the filtering module 310 may comprise a “good/bad” classifier that is trained using a similar training dataset as discussed above (i.e., comprising an existing collection of training images that meet the semantic and visual criteria 117). In another example, the filtering module 310 may comprise multiple specific detection algorithms that are each trained to explicitly look for a specific kind of anomaly. In still other examples, the filtering module 310 may be implemented as an algorithm that does not require ML-style training, such as a legacy distance algorithm(s).

As shown in FIG. 4, the filtering module 310 may receive candidate image 1 from the generative module 305 and compare it to the visual and semantic criteria 117 in any appropriate way as discussed above. If the filtering module 310 determines that candidate image 1 meets the visual and semantic criteria 117, it may store the candidate image 1 in the image database 320. If the filtering module 310 determines that candidate image 1 does not meet the visual and semantic criteria 117, it may discard it. The filtering module 310 may store each candidate image that it identifies as meeting the semantic and visual criteria 117 in the image database 320 as a test image.

In some embodiments, the filtering module 310 may be omitted and each candidate image generated by the generative module 305 may be stored directly in the image database 320 as a test image.

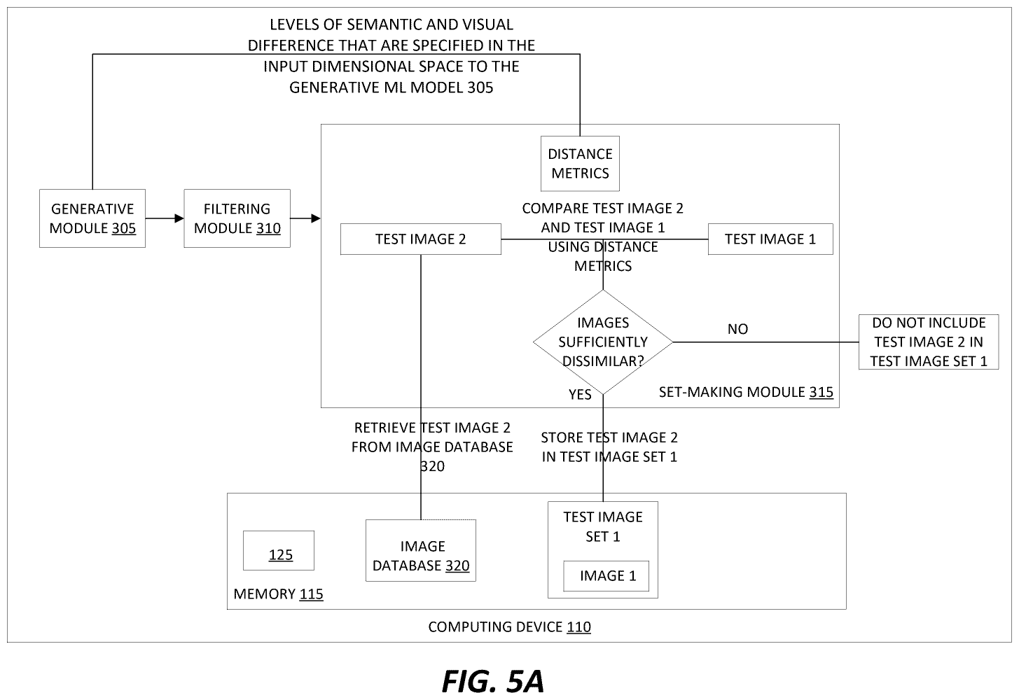

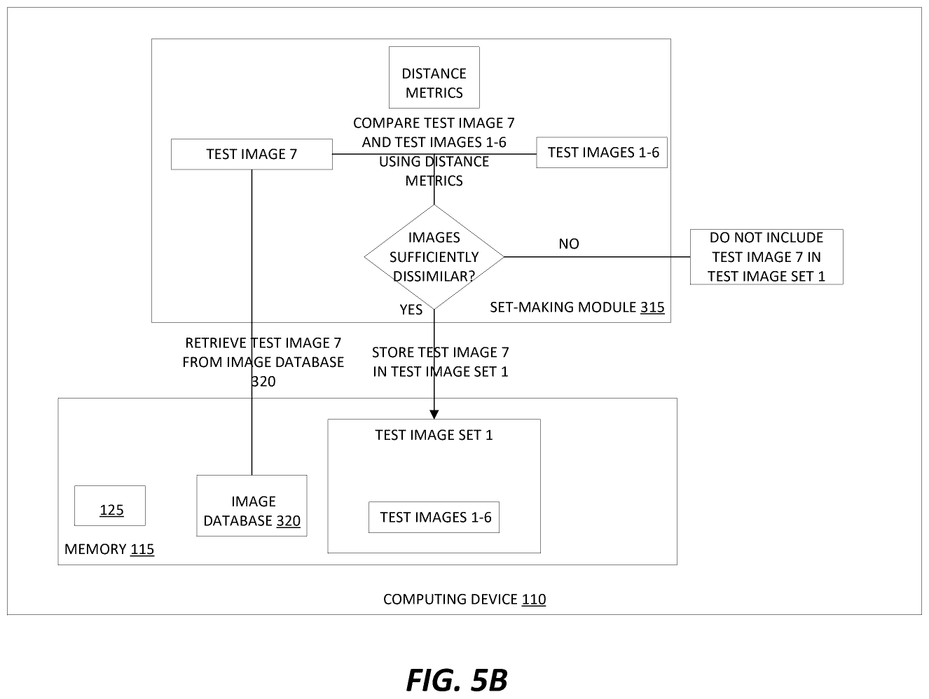

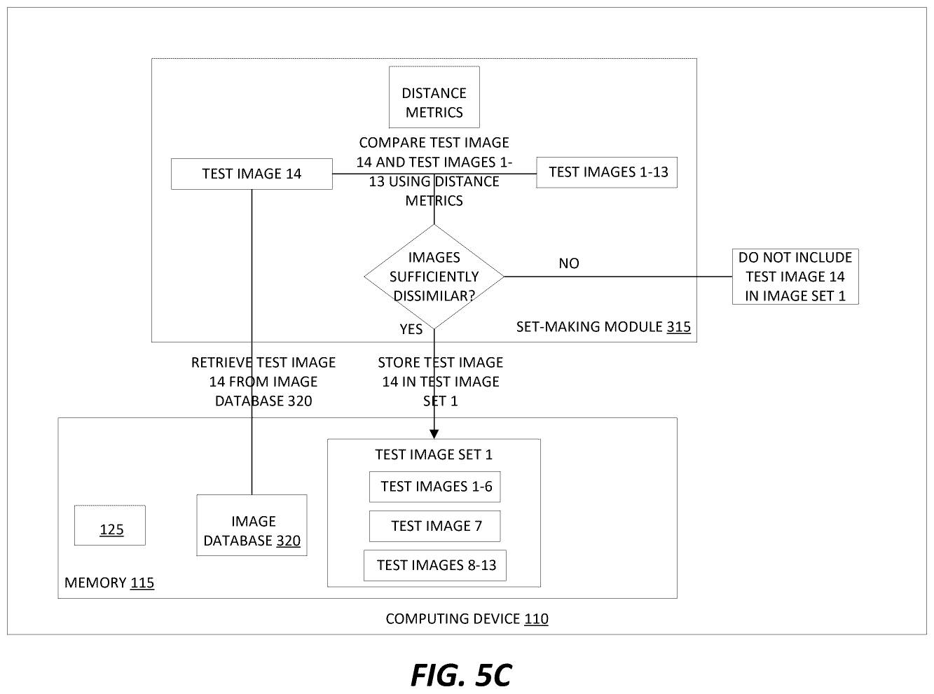

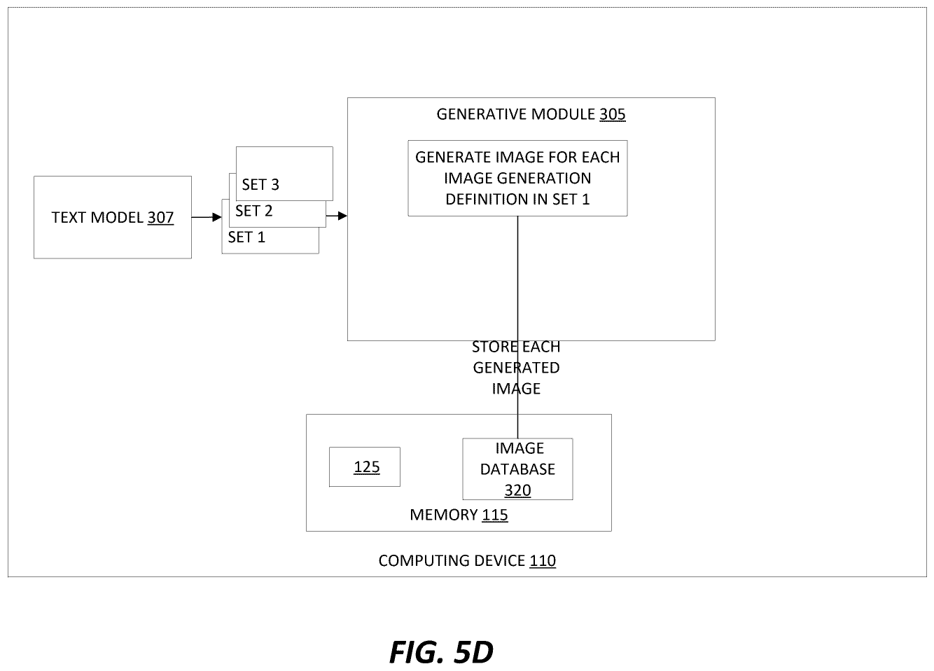

FIGS. 5A-5C illustrates the operation of the set making module 315. The set making module 315 may generate a test image set by selecting test images from the image database 320, and comparing each selected test image to any test images that are currently in the test image set being generated to determine whether the selected test image is sufficiently distinct from other test images in the image set. More specifically, as the set making module 315 selects test images, it may ensure that it selects a key test image for every X distractor test images. In some embodiments, the set making module 315 may utilize the ratio of key images to distractor images specified in the input dimensional space to the generative module 305 to ensure that it is selecting the correct number of distractor images for every key image selected. In some embodiments, the set making module 315 may comprise any appropriate algorithm (such as a stochastic optimization algorithm) that can make use of the different levels of semantic and visual difference that are specified in the input dimensional space to the generative module 305 to compare test images. For example, the set making module 315 may receive (as distance metrics as shown in FIG. 5A) the base level of semantic and visual difference that must exist between all images, the first heightened level of semantic and visual difference that must exist between key images and distractor images, and the second heightened level of semantic and visual difference that must exist between key images from the generative module 305.

In some embodiments, the set making module 315 may derive the different levels of semantic and visual difference utilized by the generative module 305 using embeddings of the generative module 305. More specifically, the features learned by the generative module 305 may be transformed into a more compact and condensed vector representation, commonly referred to as an “embedding.” The embeddings of the generative module 305 can be used to train the set making module 315 using a process called transfer learning. In transfer learning, a pre-trained model is used as a starting point to learn a new task or improve an existing model's performance. The pre-trained model is usually trained on a large dataset to recognize patterns in data, and this model (or a different model) can then be fine-tuned on a smaller, related dataset to perform a specific task. Here, the embeddings from the generative module 305 (the pre-trained model in this example) may be “transferred” to the set making module 315 by training the set making module 315 on top of these embeddings or performing vector operations such as a similarity search on the given embeddings. The set making module 315 can then be fine-tuned on a smaller, related dataset to generate image sets as discussed herein.

As shown in FIG. 5A, the set making module 315 may begin generating test image set 1, and may select a first test image, test image 1, which is a distractor test image. As test image 1 is the first selected image, it may be automatically included in the test image set 1. The set making module 315 may then select (i.e., retrieve from image database 320) test image 2, which is also a distractor test image. The set making module 315 may compare test image 2 and test image 1 to ensure that test image 2 has at least the base level of semantic and visual difference from test image 1. If test image 2 does have at least the base level of semantic and visual difference from test image 1, the set making module 315 may include it in test image set 1. Otherwise, test image 2 will not be included in test image set 1.

Referring now to FIG. 5B, after adding test images 1-6 (all distractor test images) to test image set 1, the set making module 315 may then select (i.e., retrieve from image database 320) test image 7, which is a key test image. The set making module 315 may compare test image 7 to test images 1-6 to ensure that test image 7 has at least the first heightened level of semantic and visual difference from each of test images 1-6. In the example of FIG. 5B, test image 7 does have at least the first heightened level of semantic and visual difference from each of test images 1-6 and so is added to test image set 1.

Referring now to FIG. 5C, after adding test images 8-13 (all distractor test images) to test image set 1, the set making module 315 may then select (i.e., retrieve from image database 320) test image 14, which is a key test image. The set making module 315 may compare test image 14 to test images 1-6 and test images 8-13 to ensure that test image 7 has at least the first heightened level of semantic and visual difference from each of test images 1-6 and test images 8-13. The set making module 315 may also compare test image 14 to test image 7 to ensure that test image 14 has at least the second heightened level of difference from test image 7. In the example of FIG. 5C, test image 14 does have at least the first heightened level of semantic and visual difference from each of test images 1-6 and test images 8-13, and does have at least the second heightened level of semantic and visual difference from test image 7. Therefore, the set making module 315 may add test image 14 to test image set 1.

It should be noted that if a key test image does not have at least the first heightened level of semantic and visual difference from the distractor test images currently in the test image set or at least the second heightened level of semantic and visual difference from the other key images currently in the test image set (resulting in the key test image not being added to the test image set), another key test image may be selected and added (if it meets the difference requirements) before any other distractor test images are added to the test image set. However, the test image set does not need to be built in any particular order. For example, if a test image set is to include 500 images with every e.g., 10^{th} image being a key image, and upon reaching 470 images, the set making module 315 determines that there are currently only 20 key images, it may only select key images for the last 30 images so that the appropriate ratio of key images to distractor images is achieved.

Although only one test image set is shown in FIGS. 5A-5C, the set making module 315 may generate a number of test image sets using the test images in image database 320. Once the test image sets are generated, they may be stored in memory 115, and used to assess a patient. For example, a first test image set may be used during a first assessment, and a new test image set may be used for each subsequent assessment, allowing for longitudinal tracking of progression, such as response to a therapy or treatment. Having a number of test image sets available also increases the precision of detection/diagnosis since there is more statistical power behind such detection/diagnosis due to both the raw increase in evidence and the ability to repeat assessments over different time periods (either aggregating multiple samples, each with random noise (e.g. “good” days, and “bad” days), in the same time period; or taking periodic samples to track change over time).

Although described above as a ML model, this is not a limitation and in some embodiments the set making module 315 may be implemented as an algorithm that does not require ML-style training, and may be tuned/optimized by hand. For example, the set making module 315 may comprise a greedy search algorithm.

Although illustrated as generating and storing test image sets for future use, this is also not a limitation. In some embodiments, the image set generation module 125 may be executed to generate one or more test image sets on an ad-hoc basis e.g., during an assessment itself. For example, a patient may begin an assessment and the generative module 305 may begin generating candidate images. As each candidate image is verified as a test image by the filtering module 310, the test image may be provided to the set making module 315 instead of (or in addition to) being stored in image database 320. As the set making module 315 confirms that each test image it receives meets the distance metrics (as discussed above), it may be displayed directly to the patient, instead of (or in addition to) being added to a test image set. It should be noted that in such embodiments, the set making module 315 may confirm that each test image it receives meets the distance metrics with respect to test images that have previously been displayed to the patient. It should further be noted that in such embodiments, the order in which test images are generated/displayed does matter. For example, if an assessment requires every 10^{th} image to be a key image, the generative module 305 may be configured to generate a key image as every 10^{th} image it generates. In some embodiments, the generative module 305 may be configured to wait for confirmation that a candidate image it generated was verified by the filtering module 310 and the set making module 315 and displayed to the patient, before generating a subsequent candidate image. In this way, if a candidate image of a certain type (distractor or key) was not displayed to the user (e.g., because it did not meet the difference metric requirements), the generative module 305 can generate another image of that type, and thus maintain the order of the assessment.

In some embodiments, certain parts of the image set generation module 125 may be executed on an ad-hoc basis. For example, the generative module 305 may generate candidate images and the filtering module 310 may verify the candidate images as test images and then store the test images in the image database 320. Once a patient's assessment begins, the set making module 315 may begin retrieving test images from the image database 320 and analyzing them as discussed above. For each test image that meets the distance metrics (as discussed above), the set making module 315 may display the test image directly to the patient, instead of (or in addition to) being added to a test image set. It should be noted that in such embodiments, the order in which test images are generated/displayed does matter, and thus if an assessment requires every 10^{th} image to be a key image, every 10^{th} test image retrieved from the image database 320 by the set making module 315 may be a key test image.

In some embodiments, the image set generation module 125 may generate test image sets by analyzing the visual and semantic criteria 117 and generating multiple sets of image generation definitions, based on which the generative module 305 may generate test images. Referring to FIG. 5D, the image set generation module 125 may comprise a text model 307 and the generative module 305. The text model 307 may be any appropriate ML model that takes as input the visual and semantic criteria 117 and generates multiple sets of image generation definitions, where each image generation definition in a set of image generation definitions includes a value for each characteristic of the visual and semantic criteria 117. The text model 307 may be trained to generate, using the characteristics of the visual and semantic criteria 117 (i.e., the input dimensional space), semantic descriptions of scenes (images) that are “far away” from each other (i.e., meet a distance metric/threshold), where each scene corresponds to an image generation definition from a set of image generation definitions. In some embodiments, the text model 307 may comprise natural language processing (NLP) models/utilize NLP techniques to enable the conversion of input text descriptions into corresponding visual representations.

For example, a first set (referred to as set 1) may include three image generation definitions: “green plastic tree in center of image,” “red metal train in center of image” and “photographs of crowds of people.” A second set (referred to as set 2) may include three image generation definitions: “blue metal can in center of image,” “purple wooden bear in center of image” and “photographs of animals in the desert.”

The generative module 305 may generate an image for each image generation definition in set 1, thus creating a test image set. This can be repeated multiple times for one set, and/or for multiple sets, as needed. In this way, the image set generation module 125 may create a test image set by manipulating the visual and semantic criteria 117 alone, instead of relying on the generative module 305, the filtering module 310 and the set making module 315 to generate test images based on the visual and semantic criteria 117 and certain distance metrics, as discussed hereinabove.

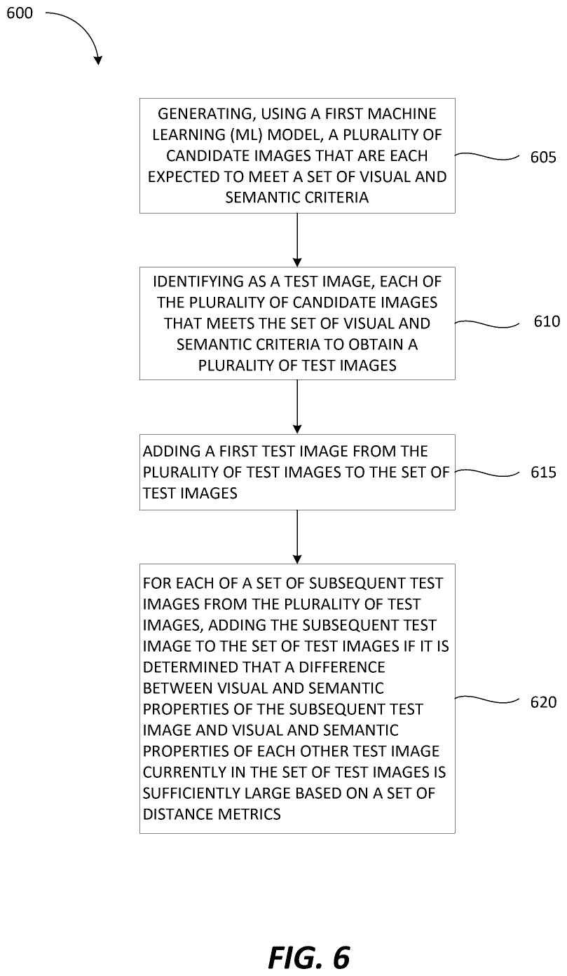

FIG. 6 is a flow diagram depicting a method 600 for generating image sets for cognitive assessment, according to some embodiments of the present disclosure. Method 600 may be performed by processing logic that may include hardware (e.g., circuitry, dedicated logic, programmable logic, a processor, a processing device, a central processing unit (CPU), a system-on-chip (SoC), etc.), software (e.g., instructions and/or an application that is running/executing on a processing device), firmware (e.g., microcode), or a combination thereof. In some embodiments, method 600 may be performed by processing device 120 (executing image set generation module 125), as shown in FIGS. 1 and 2.

With reference to FIG. 6, method 600 illustrates example functions used by various embodiments. Although specific function blocks (“blocks”) are disclosed in method 600, such blocks are examples. That is, embodiments are well suited to performing various other blocks or variations of the blocks recited in method 600. It is appreciated that the blocks in method 600 may be performed in an order different than presented, and that not all of the blocks in method 600 may be performed.

The processing device 120 may generate an input dimensional space for the generative module 305 based on the visual and semantic criteria 117 and other factors. More specifically, the processing device 120 may generate an input dimensional space that specifies generation of images that meet the visual and semantic criteria 117, specifies a base level of semantic and visual difference that must exist between all images, specifies a first heightened level of semantic and visual difference that must exist between key images and distractor images, and specifies a second heightened level of semantic and visual difference that must exist between key images. In some embodiments, the processing device 120 may include as part of the input dimensional space, a list of candidate objects of interest which the generative module 305 will also use to create images. Because it is important for images used in a test image set to have a base level of visual and semantic difference from each other, the processing device 120 may specify (e.g., as text that is included within the input dimensional space) that when creating an new image using an object of interest that has been used to create one or more previous images, the generative module 305 should change certain visual properties of the object of interest (e.g., color, position, orientation) when creating the new image so that the new image differs sufficiently from the one or more previous images that include the same object of interest. The processing device 120 may also specify input a ratio of key images to distractor images in the input dimensional space so that the generative module 305 can generate a candidate key image for every X candidate distractor images it generates based on the ratio. The ratio of key images to distractor images may correspond to the ratio of key images to distractor images required in a test image set.

Based on the input dimensional space, at block 605 the generative module 305 may begin generating candidate images (including distractor images and key images) that are expected to meet the visual and semantic criteria 117 as well as the various levels of semantic and visual difference that must exist (as discussed hereinabove). The generative module 305 may identify each candidate image it generates as a key image or a distractor image.

As the generative module 305 generates candidate images, at block 610 each candidate image may be analyzed by the filtering module 310 to determine whether the candidate image meets the visual and semantic criteria 117. The filtering module 310 may store each candidate image that it identifies as meeting the semantic and visual criteria 117 in the image database 320 as a test image. The filtering module 310 may discard each candidate image that it determines does not meet the semantic and visual criteria 117.

Referring also to FIG. 5A, at block 615 the set making module 315 may begin generating test image set 1, and may select a first test image, test image 1, which is a distractor test image. As test image 1 is the first selected image, it may be automatically included in the test image set 1. At block 620, the set making module 315 may continue generating the test image set 1 by selecting subsequent test images from the image database 320, and comparing each selected test image to any test images that are currently in the test image set being generated to determine whether the selected test image is sufficiently distinct from other test images in the image set. Continuing the example of FIG. 5A, the set making module 315 may then select (i.e., retrieve from image database 320) test image 2, which is also a distractor test image. The set making module 315 may compare test image 2 and test image 1 to ensure that test image 2 has at least the base level of semantic and visual difference from test image 1. If test image 2 does have at least the base level of semantic and visual difference from test image 1, the set making module 315 may include it in test image set 1. Otherwise, test image 2 will not be included in test image set 1.

As the set making module 315 selects test images, it may ensure that it selects a key test image for every X distractor test images. In some embodiments, the set making module 315 may utilize the ratio of key images to distractor images specified in the input dimensional space to the generative module 305 to ensure that it is selecting the correct number of distractor images for every key image selected. In some embodiments, the set making module 315 may comprise any appropriate algorithm (such as a stochastic optimization algorithm) that can make use of the different levels of semantic and visual difference that are specified in the input dimensional space to the generative module 305 to compare test images. For example, the set making module 315 may receive (as distance metrics as shown in FIG. 5A) the base level of semantic and visual difference that must exist between all images, the first heightened level of semantic and visual difference that must exist between key images and distractor images, and the second heightened level of semantic and visual difference that must exist between key images from the generative module 305.

Referring also to FIG. 5B, after adding test images 1-6 (all distractor test images) to test image set 1, the set making module 315 may then select (i.e., retrieve from image database 320) test image 7, which is a key test image. The set making module 315 may compare test image 7 to test images 1-6 to ensure that test image 7 has at least the first heightened level of semantic and visual difference from each of test images 1-6. In the example of FIG. 5B, test image 7 does have at least the first heightened level of semantic and visual difference from each of test images 1-6 and so is added to test image set 1.

Referring also to FIG. 5C, after adding test images 8-13 (all distractor test images) to test image set 1, the set making module 315 may then select (i.e., retrieve from image database 320) test image 14, which is a key test image. The set making module 315 may compare test image 14 to test images 1-6 and test images 8-13 to ensure that test image 7 has at least the first heightened level of semantic and visual difference from each of test images 1-6 and test images 8-13. The set making module 315 may also compare test image 14 to test image 7 to ensure that test image 14 has at least the second heightened level of difference from test image 7. In the example of FIG. 5C, test image 14 does have at least the first heightened level of semantic and visual difference from each of test images 1-6 and test images 8-13, and does have at least the second heightened level of semantic and visual difference from test image 7. Therefore, the set making module 315 may add test image 14 to test image set 1.

It should be noted that if a key test image does not have at least the first heightened level of semantic and visual difference from the distractor test images currently in the test image set or at least the second heightened level of semantic and visual difference from the other key images currently in the test image set (resulting in the key test image not being added to the test image set), another key test image may be selected and added (if it meets the difference requirements) before any other distractor test images are added to the test image set. However, the test image set does not need to be built in any particular order. For example, if a test image set is to include 500 images with every e.g., 10^{th} image being a key image, and upon reaching 470 images, the set making module 315 determines that there are currently only 20 key images, it may only select key images for the last 30 images so that the appropriate ratio of key images to distractor images is achieved.

Although only one test image set is shown in FIGS. 5A-5C, the set making module 315 may generate a number of test image sets using the test images in image database 320. Once the test image sets are generated, they may be stored in memory 115, and used to assess a patient. For example, a first test image set may be used during a first assessment, and a new test image set may be used for each subsequent assessment, allowing for longitudinal tracking of progression, such as response to a therapy or treatment. Having a number of test image sets available also increases the precision of detection/diagnosis since there is more statistical power behind such detection/diagnosis due to both the raw increase in evidence and the ability to repeat assessments over different time periods (either aggregating multiple samples, each with random noise (e.g. “good” days, and “bad” days), in the same time period; or taking periodic samples to track change over time).

Although illustrated as generating and storing test image sets for future use, this is also not a limitation. In some embodiments, the image set generation module 125 may be executed to generate one or more test image sets on an ad-hoc basis e.g., during an assessment itself. For example, a patient may begin an assessment and the generative module 305 may begin generating candidate images. As each candidate image is verified as a test image by the filtering module 310, the test image may be provided to the set making module 315 instead of (or in addition to) being stored in image database 320. As the set making module 315 confirms that each test image it receives meets the distance metrics (as discussed above), it may be displayed directly to the patient, instead of (or in addition to) being added to a test image set. While test images are being generated during an assessment, the computing device 110 may be in communication with a monitoring device (not shown) which may record electrical signals corresponding to cognitive activity of the patient during the assessment. For example, the monitoring device may be an EEG device that records electrical activity of the patient's brain using one or more electrodes. In other examples, the monitoring device may be a functional near-infrared spectroscopy (fNIRS) device or a magnetoencephalography (MEG) device. It should be noted that in such embodiments, the set making module 315 may confirm that each test image it receives meets the distance metrics with respect to test images that have previously been displayed to the patient. It should further be noted that in such embodiments, the order in which test images are generated/displayed does matter. For example, if an assessment requires every 10^{th} image to be a key image, the generative module 305 may be configured to generate a key image as every 10^{th} image it generates. In some embodiments, the generative module 305 may be configured to wait for confirmation that a candidate image it generated was verified by the filtering module 310 and the set making module 315 and displayed to the patient, before generating a subsequent candidate image. In this way, if a candidate image of a certain type (distractor or key) was not displayed to the user (e.g., because it did not meet the difference metric requirements), the generative module 305 can generate another image of that type, and thus maintain the order of the assessment.

In some embodiments, certain parts of the image set generation module 125 may be executed on an ad-hoc basis. For example, the generative module 305 may generate candidate images and the filtering module 310 may verify the candidate images as test images and then store the test images in the image database 320. Once a patient's assessment begins, the set making module 315 may begin retrieving test images from the image database 320 and analyzing them as discussed above. For each test image that meets the distance metrics (as discussed above), the set making module 315 may display the test image directly to the patient, instead of (or in addition to) being added to a test image set. It should be noted that in such embodiments, the order in which test images are generated/displayed does matter, and thus if an assessment requires every 10^{th} image to be a key image, every 10^{th} test image retrieved from the image database 320 by the set making module 315 may be a key test image.

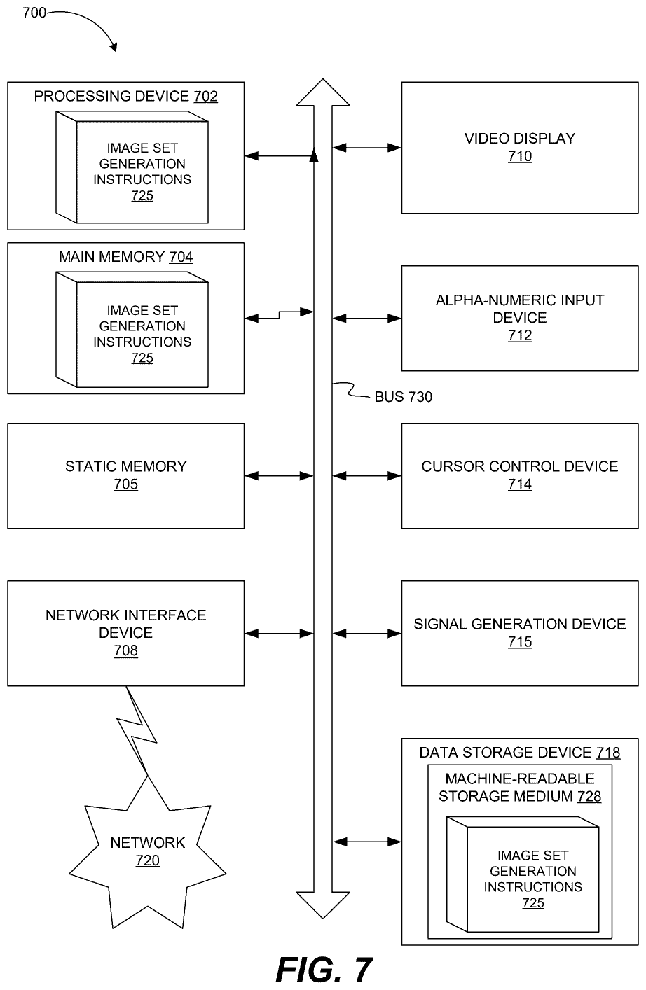

FIG. 7 is a block diagram of an example computing device 700 that may perform one or more of the operations described herein, in accordance with some embodiments. Computing device 700 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 700 may include a processing device (e.g., a general purpose processor, a PLD, etc.) 702, a main memory 704 (e.g., synchronous dynamic random access memory (DRAM), read-only memory (ROM)), a static memory 705 (e.g., flash memory and a data storage device 718), which may communicate with each other via a bus 730.

Processing device 702 may be provided by one or more general-purpose processing devices such as a microprocessor, central processing unit, or the like. In an illustrative example, processing device 702 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 702 may also include one or more special-purpose processing devices such as an application specific integrated circuit (ASIC), a field programmable gate array (FPGA), a digital signal processor (DSP), network processor, or the like. The processing device 702 may be configured to execute the operations described herein, in accordance with one or more aspects of the present disclosure, for performing the operations and steps discussed herein.

Computing device 700 may further include a network interface device 708 which may communicate with a communication network 720. The computing device 700 also may include a video display unit 710 (e.g., a liquid crystal display (LCD) or a cathode ray tube (CRT)), an alphanumeric input device 712 (e.g., a keyboard), a cursor control device 714 (e.g., a mouse) and an acoustic signal generation device 715 (e.g., a speaker). In one embodiment, video display unit 710, alphanumeric input device 712, and cursor control device 714 may be combined into a single component or device (e.g., an LCD touch screen).

Data storage device 718 may include a computer-readable storage medium 728 on which may be stored one or more sets of image set generation instructions 725 that may include instructions for one or more components, agents, and/or applications for carrying out the operations described herein, in accordance with one or more aspects of the present disclosure. image set generation instructions 725 may also reside, completely or at least partially, within main memory 704 and/or within processing device 702 during execution thereof by computing device 700, main memory 704 and processing device 702 also constituting computer-readable media. The image set generation instructions 725 may further be transmitted or received over a communication network 720 via network interface device 708.

While computer-readable storage medium 728 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

Unless specifically stated otherwise, terms such as “allocating,” “detecting,” “migrating,” or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and may not necessarily have an ordinal meaning according to their numerical designation.

Examples described herein also relate to an apparatus for performing the operations described herein. This apparatus may be specially constructed for the required purposes, or it may include a general purpose computing device selectively programmed by a computer program stored in the computing device. Such a computer program may be stored in a computer-readable non-transitory storage medium.

The methods and illustrative examples described herein are not inherently related to any particular computer or other apparatus. Various general purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware--for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. § 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

The foregoing description, for the purpose of explanation, has been described with reference to specific embodiments. However, the illustrative discussions above are not intended to be exhaustive or to limit the present disclosure to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the present disclosure is not to be limited to the details given herein, but may be modified within the scope and equivalents of the appended claims.

What is claimed is:

1. A method comprising:
generating, using a first machine learning (ML) model, a plurality of candidate images that are each expected to meet a set of visual and semantic criteria;
identifying as a test image, each of the plurality of candidate images that meets the set of visual and semantic criteria to obtain a plurality of test images; and
generating by a processing device, using a second ML model, a set of test images using the plurality of test images by:
adding a first test image from the plurality of test images to the set of test images; and
for each of a set of subsequent test images from the plurality of test images, adding the subsequent test image to the set of test images if it is determined that a difference between visual and semantic properties of the subsequent test image and visual and semantic properties of each other test image currently in the set of test images meets a threshold based on a set of distance metrics.

2. The method of claim 1, further comprising:
displaying, as they are added to the set of test images, the first test image and each of the set of subsequent test images that is added to the set of test images.

3. The method of claim 1, further comprising:
building an input dimensional space for the first ML model; and
defining within the input dimensional space, the set of distance metrics, wherein the set of distance metrics comprises:
a first minimum distance between visual and semantic properties of test images that are key images and visual and semantic properties of test images that are distractor images;
a second minimum distance between visual and semantic properties of test images that are key images; and
a third minimum distance between visual and semantic properties of all test images.

4. The method of claim 1, wherein the set of visual and semantic criteria include:
a generic and non-descript background;
a unitary object of interest whose semantics can be determined;
a restriction that the unitary object of interest cannot contain human or animal faces;
a restriction on the subject matter of the unitary object of interest; and
a position and orientation of the unitary object of interest.

5. The method of claim 1, wherein generating the plurality of candidate images comprises:
generating each of the plurality of candidate images using a candidate object of interest from a predefined list of candidate objects of interest.

6. The method of claim 5, wherein generating a candidate image of the plurality of candidate images using a candidate object of interest comprises:
determining whether the candidate object of interest has been previously used to generate any of the plurality of candidate images;
in response to determining that the candidate object of interest has been previously used to generate any of the plurality of candidate images, modifying one or more visual properties of the candidate object of interest; and
generating the candidate image of the plurality of images using the modified candidate object of interest.

7. The method of claim 1, wherein a third ML model identifies as a test image, each of the plurality of candidate images that meets the set of visual and semantic criteria.

8. A system comprising:
a memory; and
a processing device operatively coupled to the memory, the processing device to:
generating, using a first machine learning (ML) model, a plurality of candidate images that are each expected to meet a set of visual and semantic criteria;
identify as a test image, each of the plurality of candidate images that meets the set of visual and semantic criteria to obtain a plurality of test images; and
generate, using a second ML model, a set of test images using the plurality of test images by:
adding a first test image from the plurality of test images to the set of test images; and
for each of a set of subsequent test images from the plurality of test images, adding the subsequent test image to the set of test images if it is determined that a difference between visual and semantic properties of the subsequent test image and visual and semantic properties of each other test image currently in the set of test images meets a threshold based on a set of distance metrics.

9. The system of claim 8, wherein the processing device is further to:
display, as they are added to the set of test images, the first test image and each of the set of subsequent test images that is added to the set of test images.

10. The system of claim 8, wherein the processing device is further to:
build an input dimensional space for the first ML model; and
define within the input dimensional space, the set of distance metrics, wherein the set of distance metrics comprises:
a first minimum distance between visual and semantic properties of test images that are key images and visual and semantic properties of test images that are distractor images;
a second minimum distance between visual and semantic properties of test images that are key images; and
a third minimum distance between visual and semantic properties of all test images.

11. The system of claim 8, wherein the set of visual and semantic criteria include:
a generic and non-descript background;
a unitary object of interest whose semantics can be determined;
a restriction that the unitary object of interest cannot contain human or animal faces;
a restriction on the subject matter of the unitary object of interest; and
a position and orientation of the unitary object of interest.

12. The system of claim 8, wherein to generate the plurality of candidate images, the processing device is to:
generate each of the plurality of candidate images using a candidate object of interest from a predefined list of candidate objects of interest.

13. The system of claim 12, wherein to generate a candidate image of the plurality of candidate images using a candidate object of interest, the processing device is to:
determine whether the candidate object of interest has been previously used to generate any of the plurality of candidate images;
in response to determining that the candidate object of interest has been previously used to generate any of the plurality of candidate images, modify one or more visual properties of the candidate object of interest; and
generate the candidate image of the plurality of images using the modified candidate object of interest.

14. The system of claim 8, wherein the processing device uses a third ML model to identify as a test image, each of the plurality of candidate images that meets the set of visual and semantic criteria.

15. A non-transitory computer-readable medium having instructions stored thereon which, when executed by a processing device, cause the processing device to:
generate, using a first machine learning (ML) model, a plurality of candidate images that are each expected to meet a set of visual and semantic criteria;
identify as a test image, each of the plurality of candidate images that meets the set of visual and semantic criteria to obtain a plurality of test images; and
generate, using a second ML model, a set of test images using the plurality of test images by:
adding a first test image from the plurality of test images to the set of test images; and
for each of a set of subsequent test images from the plurality of test images, adding the subsequent test image to the set of test images if it is determined that a difference between visual and semantic properties of the subsequent test image and visual and semantic properties of each other test image currently in the set of test images meets a threshold based on a set of distance metrics.

16. The non-transitory computer-readable medium of claim 15, wherein the processing device is further to:
display, as they are added to the set of test images, the first test image and each of the set of subsequent test images that is added to the set of test images.

17. The non-transitory computer-readable medium of claim 15, wherein the processing device is further to:
build an input dimensional space for the first ML model; and
define within the input dimensional space, the set of distance metrics, wherein the set of distance metrics comprises:
a first minimum distance between visual and semantic properties of test images that are key images and visual and semantic properties of test images that are distractor images;
a second minimum distance between visual and semantic properties of test images that are key images; and
a third minimum distance between visual and semantic properties of all test images.

18. The non-transitory computer-readable medium of claim 15, wherein the set of visual and semantic criteria include:
a generic and non-descript background;
a unitary object of interest whose semantics can be determined;
a restriction that the unitary object of interest cannot contain human or animal faces;
a restriction on the subject matter of the unitary object of interest; and
a position and orientation of the unitary object of interest.

19. The non-transitory computer-readable medium of claim 15, wherein to generate the plurality of candidate images, the processing device is to:
generate each of the plurality of candidate images using a candidate object of interest from a predefined list of candidate objects of interest.

20. The non-transitory computer-readable medium of claim 19, wherein to generate a candidate image of the plurality of candidate images using a candidate object of interest, the processing device is to:
determine whether the candidate object of interest has been previously used to generate any of the plurality of candidate images;
in response to determining that the candidate object of interest has been previously used to generate any of the plurality of candidate images, modify one or more visual properties of the candidate object of interest; and
generate the candidate image of the plurality of images using the modified candidate object of interest.
